(19) United States
(12) Patent Application Publication
(10) Pub. No.: US 2025/0354893 A1
(43) Pub. Date: Nov. 20, 2025

(54) GAS SENSOR

(71) Applicant: Niterra Co., Ltd., Nagoya-shi (JP)

(72) Inventors: Yusuke ANDO, Nagoya-shi (JP); Kensuke MIZUTANI, Nagoya-shi (JP); Yasuhiro FUJII, Nagoya-shi (JP)

(73) Assignee: Niterra Co., Ltd., Nagoya-shi (JP)

(21) Appl. No.: 19/042,578

(22) Filed: Jan. 31, 2025

(30) Foreign Application Priority Data
May 17, 2024 (JP) 2024-080719

(51) Int. Cl. G01M 15/10

(52) U.S. Cl. CPC G01M 15/104

(57) ABSTRACT

A gas sensor comprising: a sensor element; a metal shell; a holder having a first element hole; a sleeve having a second element hole; and filling powder, wherein at least one of the holder and the sleeve has a flat surface contacting with the filling powder, and a protrusion protruding from the flat surface toward the filling powder and surrounding the first element hole or the second element hole, the protrusion is tapered toward the filling powder, as seen from a direction D perpendicular to a main surface of the sensor element, and a boundary portion BR where corners parallel to the axial-line-O direction of the sensor element abut on the protrusion is located between a top portion of the protrusion and a base portion.

### CROSS-REFERENCE TO RELATED APPLICATION

This application claims the benefit of priority to Japanese Patent Application No. 2024-080719 filed on May 17, 2024, the content of which is incorporated herein by reference in its entity.

### FIELD OF THE INVENTION

The present invention relates to a gas sensor including a sensor element for detecting the concentration of a detection target gas.

### BACKGROUND OF THE INVENTION

As a gas sensor for detecting the concentration of oxygen or NOx in exhaust gas of an automobile or the like, a gas sensor having a plate-shaped sensor element is known. One of the gas sensors of this type is configured such that a periphery of a sensor element is retained by a tubular metal shell, an annular holder and an annular sleeve having element holes through which the sensor element is inserted are provided in an internal hole of the metal shell, and filling powder such as a talc ring is provided between the holder and the sleeve to fill a gap between the metal shell and the sensor element (Japanese Patent Application Laid-Open (kokai) No. 2019-138679).

Here, the sleeve is crimped so as to be pressed toward the holder, whereby the filling powder is compressed to enter a gap between the metal shell and the sensor element, thus performing a sealing function.

### PRIOR ART DOCUMENT

### Patent Document

Patent Document 1: Japanese Patent Application Laid-Open (kokai) No. 2019-138679

### Problems to be Solved by the Invention

However, in a conventional gas sensor, as shown in FIG. 8, the peripheries of a holder 1100 and a sleeve 1200 around an element hole 1100h have flat surfaces. Therefore, when filling powder 1300 is compressed via the sleeve 1200, a pressure concentrates on corners 1000e of a sensor element 1000, which have low strength, so that the sensor element 1000 might be damaged.

The reason is as follows. That is, for example, if the periphery of the holder 1100 around the element hole 1100h has a flat surface, when the filling powder 1300 is compressed, the sensor element 1000 at the periphery of the element hole 1100h is uniformly subjected to pressing forces F from the filling powder 1300. However, this causes the low-strength corners 1000e to be subjected to the pressing forces F along with the other parts, leading to damage of the corners 1000e.

Meanwhile, since the filling powder 1300 fills (seals) the gap between the metal shell and the sensor element 1000, it is difficult to weaken the entire pressing forces F.

Accordingly, an object of the present invention is to provide a gas sensor configured to suppress damage when a sensor element is attached.

### SUMMARY OF THE INVENTION

### Means For Solving The Problem

In order to solve the above problem, a gas sensor of the present invention is a gas sensor comprising: a plate-shaped sensor element extending in an axial-line direction and having a detection portion on a front end side thereof, said detection portion configured to detect target gas; a tubular metal shell surrounding and retaining a periphery of the sensor element in a radial direction; a tubular holder retained in an internal hole of the metal shell and having a first element hole which has a rectangular shape and through which the sensor element is inserted; a tubular sleeve retained in the internal hole of the metal shell on a rear end side relative to the holder, and having a second element hole which has a rectangular shape and through which the sensor element is inserted; and filling powder provided in the internal hole of the metal shell, between the holder and the sleeve, so as to fill a gap between the metal shell and the sensor element. At least one of the holder and the sleeve has a flat surface contacting the filling powder, and a protrusion protruding from the flat surface toward the filling powder and surrounding the first element hole or the second element hole. The protrusion is tapered toward the filling powder, as seen from a direction perpendicular to a main surface of the sensor element, and a boundary portion, in which corners of the sensor element extending in the axial-line direction thereof abut on the protrusion, is located between a top portion of the protrusion and a base portion where the protrusion connects to the flat surface.

In this gas sensor, when the filling powder is compressed, a pressing force from the filling powder applied to the sensor element near the boundary portion becomes a highest pressing force on a side near the top portion, and becomes a pressing force lower, at the boundary portion. Then, the pressing force is further reduced from the boundary portion toward the base portion, and becomes a lowest pressing force at the base portion and the flat surface.

Thus, the pressing force from the filling powder weakens along the axial-line direction of the corners. Therefore, the corners having a low strength are prevented from being subjected to a high pressing force that is applied to the other parts (e.g., a part where the sensor element abuts on the top portion), whereby damage when the sensor element is attached can be suppressed.

Meanwhile, in the vicinity of the top portion, the distance to the sleeve is short and the pressing force is high, so that the gap between the metal shell and the sensor element can be assuredly filled (sealed) with the filling powder.

In the gas sensor of the present invention, as seen from the direction perpendicular to the main surface of the sensor element, a relationship of c<a<b is satisfied, where a is a width of the sensor element, b is a width of the base portion where the protrusion connects to the flat surface and c is a width of the top portion of the protrusion.

In this gas sensor, it is possible to assuredly locate the boundary portion between the top portion and the base portion.

In the gas sensor of the present invention, the top portion of the protrusion may be flat.

In this gas sensor, the filling powder in a region around the sensor element can be compressed at surfaces, as compared to a case where the top portion has a steep mountain shape. Thus, the periphery of the sensor element can be stably filled with the filling powder, and the gap between the metal shell and the sensor element can be more assuredly filled (sealed).

In the gas sensor of the present invention, the protrusion may reach an end of the flat surface.

In this gas sensor, when the protrusion reaches the end of the flat surface, accordingly, the area where the filling powder in a region around the sensor element can be compressed with a high pressing force increases. Thus, the gap between the metal shell and the sensor element can be more assuredly filled (sealed).

In the gas sensor of the present invention, the gas sensor may be an oxygen sensor or a NOx sensor.

### Effects of the Invention

The present invention makes it possible to provide a gas sensor configured to suppress damage when a sensor element is attached.

### BRIEF DESCRIPTION OF THE DRAWINGS

These and other features and advantages of the present invention will become more readily appreciated when considered in connection with the following detailed description and appended drawings, wherein like designations denote like elements in the various views, and wherein:

FIG. 1 is a sectional view along a longitudinal direction of a gas sensor according to an embodiment of the present invention.

FIG. 2 is a perspective view of a sensor element.

FIG. 3 is a perspective view of a holder.

FIG. 4 is a perspective view of a sleeve.

FIG. 5 is a front view showing the positional relationship among the sensor element, the holder, and the sleeve, as seen from the direction perpendicular to the main surface of the sensor element.

FIG. 6 is a view showing the dimensions of a protrusion and the sensor element as seen from the direction perpendicular to the main surface of the sensor element.

FIG. 7 is a perspective view showing a modification of the sleeve.

FIG. 8 is a view showing a compression state of filling powder in a conventional gas sensor.

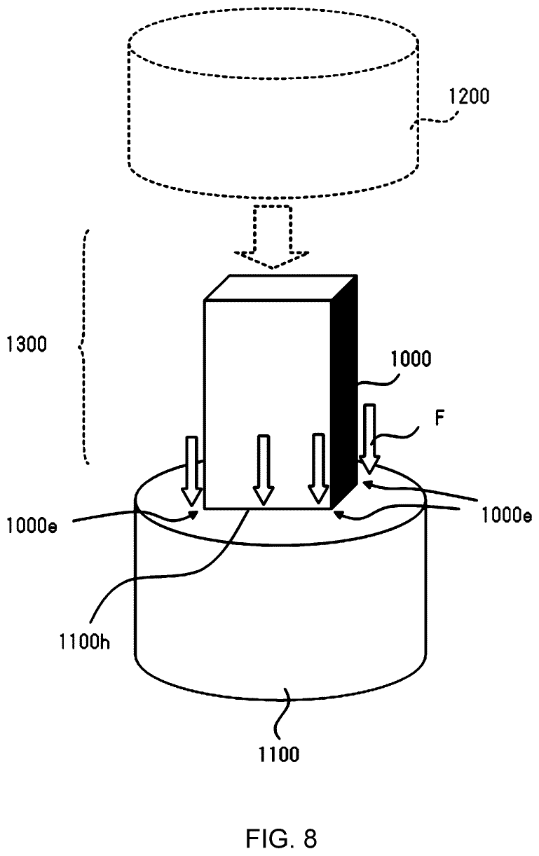

### DETAILED DESCRIPTION OF THE INVENTION

Hereinafter, an embodiment of the present invention will be described.

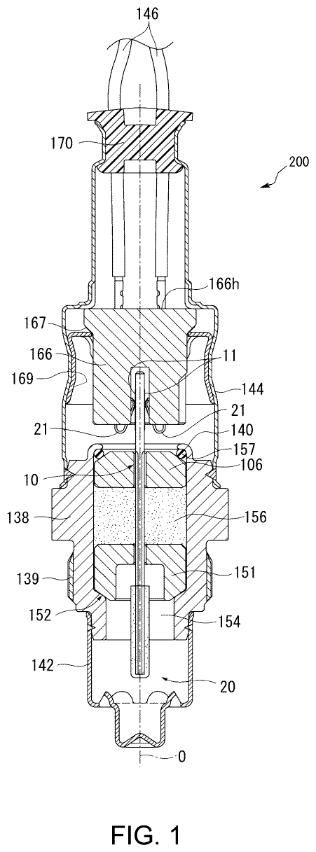

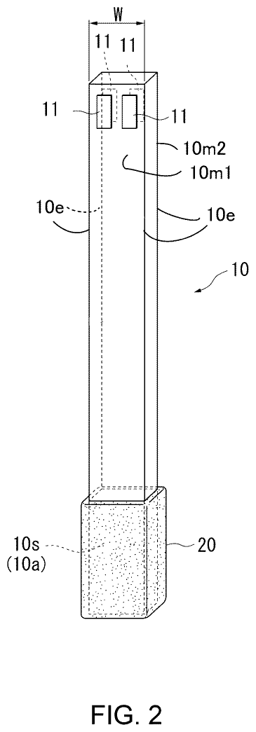

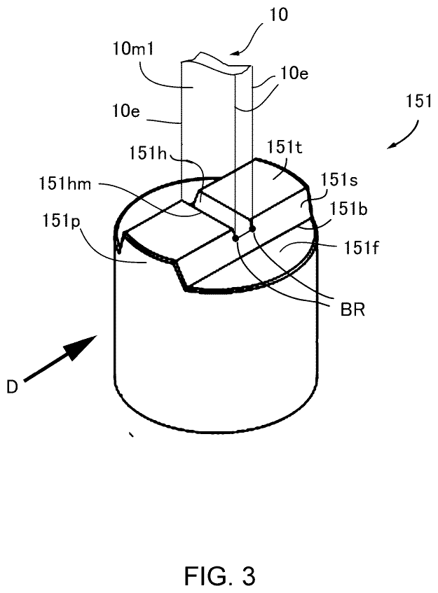

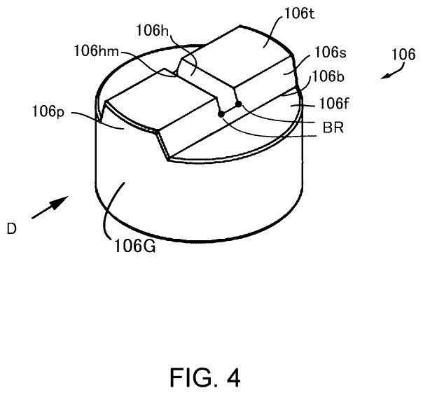

FIG. 1 is an entire sectional view along a longitudinal direction of a gas sensor (oxygen sensor) 200 according to an embodiment of the present invention. FIG. 2 is a perspective view of a sensor element 10. FIG. 3 is a perspective view of a holder 151. FIG. 4 is a perspective view of a sleeve 106.

The gas sensor 200 is an oxygen sensor for detecting the concentration of oxygen in exhaust gas of an automobile or various internal combustion engines.

In FIG. 1, the gas sensor 200 includes: a tubular metal shell 138 having, on an outer surface, a thread portion 139 to be fixed to an exhaust pipe; the sensor element 10 having a plate shape and extending in an axial-line-O direction (the longitudinal direction of the gas sensor 200, i.e., the up-down direction in the drawing); the tubular sleeve 106 made of ceramic and provided so as to surround the radial-direction periphery of the sensor element 10; the tubular holder 151 made of ceramic (alumina); a tubular separator 166 made of ceramic and provided in a state of surrounding the periphery of the rear end of the sensor element 10 inside the front end side of an insertion hole 166h penetrating in the axial-line direction; and four metal terminals 21 (only two of them are shown in FIG. 1) provided between the sensor element 10 and the separator 166.

A detection portion 10a at a front end of the sensor element 10 is covered with a porous protection layer 20 of alumina or the like (see FIG. 2).

The metal shell 138 is made of stainless steel, has a through hole 154 penetrating in the axial-line direction, and is formed substantially in a tubular shape having a ledge portion 152 protruding radially inward of the through hole 154. In the through hole 154, the sensor element 10 is placed such that a front end portion of the sensor element 10 protrudes frontward relative to the front end of the through hole 154. The ledge portion 152 is formed as an inward taper surface sloped relative to a plane perpendicular to the axial-line direction.

Inside the through hole 154 of the metal shell 138, the holder 151, a filling powder 156 (hereinafter, may be referred to as talc ring), and the aforementioned sleeve 106 are stacked in this order from the front side to the rear side so as to surround the radial-direction periphery of the sensor element 10.

A crimp packing 157 is provided between the sleeve 106 and a rear end portion 140 of the metal shell 138. The rear end portion 140 of the metal shell 138 is crimped so as to press the sleeve 106 toward the front side via the crimp packing 157.

On the other hand, as shown in FIG. 1, around the outer periphery at the front side (downward side in FIG. 1) of the metal shell 138, a single-wall protector 142 having a plurality of holes and made of metal (e.g., stainless steel) is attached by welding or the like so as to cover a protruding part of the sensor element 10.

An outer casing 144 is fixed to the outer periphery at the rear side of the metal shell 138. At an opening on the rear side (upward side in FIG. 1) of the outer casing 144, a grommet 170 made of rubber is provided, and four lead wires 146 (only two of them are shown in FIG. 1) electrically connected to the four metal terminals 21 (only two of them are shown in FIG. 1) of the sensor element 10 pass through lead wire passage holes (not shown) formed in the grommet 170.

The grommet 170 is retained inside the outer casing 144 with the grommet 170 crimped from the outer side of the outer casing 144.

The separator 166 is provided at the rear end side (upward side in FIG. 1) of the sensor element 10 protruding from the rear end portion 140 of the metal shell 138. The separator 166 is provided around a total of four electrode pads 11 (only two electrode pads are shown in FIG. 1) formed on main surfaces at the rear end side of the sensor element 10. The separator 166 is formed in a tubular shape having the insertion hole 166h penetrating in the axial-line direction, and has a flange portion 167 protruding radially outward from the outer surface. The separator 166 is held inside the outer casing 144 by the flange portion 167 contacting with the outer casing 144 via a holding member 169.

As shown in FIG. 2, the sensor element 10 has a plate shape extending in the axial-line-O direction, and a front end portion 10s thereof serves as the detection portion 10a for detecting the concentration of oxygen. The detection portion 10a is covered with the porous protection layer 20. The sensor element 10 itself has a known configuration, i.e., although not shown, has the detection portion having an oxygen-ion-permeable solid electrolyte and a pair of electrodes, and a heater portion for heating the detection portion to keep the temperature thereof constant.

Four corners 10e are formed in parallel to the axial-line-O direction of the sensor element 10.

At a rear end side on one main surface 10m1 of the sensor element 10, two electrode pads 11 are arranged in the direction of a width W, and a sensor output signal from the detection portion 10a is output from the electrode pads 11 via a lead portion (not shown). In addition, at the rear end side on another main surface 10m2 opposite to the main surface, two electrode pads 11 are arranged in the direction of the width W, whereby power is supplied to the heater portion via a lead portion (not shown).

Each electrode pad 11 has a rectangular shape elongated in the axial-line-O direction, and can be formed as a sintered body containing Pt as a main component, for example.

FIG. 3 is a perspective view of the holder 151.

The holder 151 has a tubular shape, and has a first element hole 151h which has a rectangular shape and through which the sensor element 10 is inserted.

The holder 151 has a flat surface 151f contacting the filling powder 156 (on the upper side in FIG. 3), and a protrusion 151p protruding from the flat surface 151f toward the filling powder 156 (on the upper side in FIG. 3) and surrounding the first element hole 151h.

The protrusion 151p has substantially a trapezoidal shape tapered toward the filling powder 156 (on the upper side in FIG. 3), as seen from a direction D perpendicular to the main surface 10m1 of the sensor element 10.

The flat surface 151f is formed on the radially outer side of the first element hole 151h, and the protrusion 151p is formed so as to surround the first element hole 151h from the flat surface 151f toward the radially inner side.

The protrusion 151p has a top portion 151t, and a base portion 151b at which the protrusion 151p connects to the flat surface 151f.

In this example, a slope surface 151s having a slope shape is formed between the top portion 151t and the base portion 151b. The shape of the protrusion 151p between the top portion 151t and the base portion 151b is not limited and may be a stepped shape, for example.

In this example, the top portion 151t is flat (surface).

A peripheral edge 151h m at which the first element hole 151h intersects the protrusion 151p has a contour that extends in parallel to the main surface 10m1, at the top portion 151t, then extends toward the front end side in the axial-line-O direction along the slope surface 151s, and extends in the direction D from a certain position on the slope surface 151s.

Thus, a boundary portion BR (BR also corresponds to the peripheral edge 151hm) where the four corners 10e (three of them are shown in FIG. 3) of the sensor element 10 abut on the protrusion 151p in a state in which the sensor element 10 is inserted through the first element hole 151h, is located at a certain position on the slope surface 151s, i.e., between the top portion 151t and the base portion 151b. The reason will be described later.

FIG. 4 is a perspective view of the sleeve 106. The sleeve 106 has the same configuration as the holder 151, and therefore the overview thereof will be described.

Specifically, the sleeve 106 has a tubular shape, and has a second element hole 106h which has a rectangular shape and through which the sensor element 10 is inserted.

The sleeve 106 has a flat surface 106f contacting with the filling powder 156 (on the upper side in FIG. 4), and a protrusion 106p protruding from the flat surface 106f toward the filling powder 156 (on the upper side in FIG. 4) and surrounding the second element hole 106h.

The protrusion 106p has substantially a trapezoidal shape tapered toward the filling powder 156 (on the upper side in FIG. 4), as seen from the direction D perpendicular to the main surface 10m1 of the sensor element 10.

The flat surface 106f is formed on the radially outer side of the second element hole 106h, and the protrusion 106p is formed so as to surround the second element hole 106h from the flat surface 106f toward the radially inner side.

The protrusion 106p has a top portion 106t, and a base portion 106b at which the protrusion 106p connects to the flat surface 106f. In this example, a slope surface 106s having a slope shape is formed between the top portion 106t and the base portion 106b.

A peripheral edge 106h m at which the second element hole 106h intersects the protrusion 106p has a contour similar to that of the peripheral edge 151hm of the holder 151, and similarly, a boundary portion BR (BR also corresponds to the peripheral edge 106hm) where the four corners 10e (not shown) of the sensor element 10 abut on the protrusion 106p is located at a certain position on the slope surface 106s, i.e., between the top portion 106t and the base portion 106b.

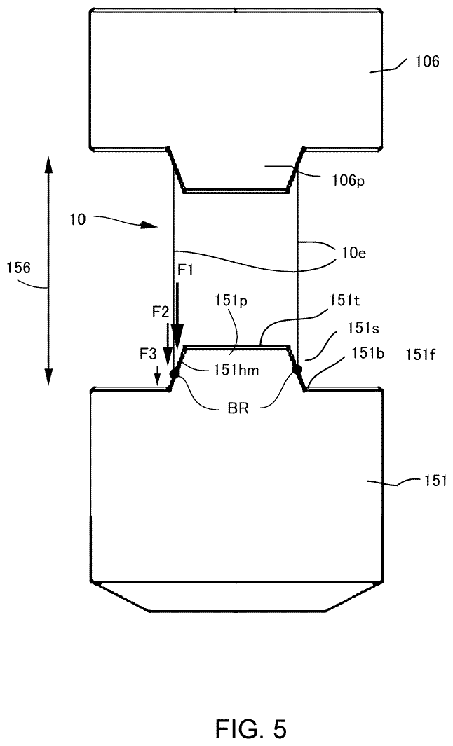

Next, with reference to FIG. 5, the reason why the boundary portion BR is located between the top portion 151t, 106t and the base portion 151b, 106b, will be described.

FIG. 5 is a front view showing the positional relationship among the sensor element 10, the holder 151, and the sleeve 106, as seen from the direction D perpendicular to the main surface 10m1 of the sensor element 10.

Focusing on the sensor element 10 and the holder 151, the four corners 10e of the sensor element 10 abut on (adjoin) the protrusion 151p at the boundary portion BR (BR also corresponds to the peripheral edge 151h m). The boundary portion BR is located at a certain position (between the top portion 151t and the base portion 151b) on the slope surface 151s.

Therefore, when the filling powder 156 is compressed via the sleeve 106, a pressing force from the filling powder 156 applied to the sensor element 10 near the boundary portion BR becomes a highest pressing force F1 on a side near the top portion 151t, and becomes a pressing force F2 lower than F1, at the boundary portion BR. Then, the pressing force is further reduced from the boundary portion BR toward the base portion 151b, and becomes a lowest pressing force F3 at the base portion 151b and the flat surface 151f.

This is because the top portion 151t is at a distance closest to the sleeve 106, and thus is subjected to a highest compression load of the filling powder 156.

Thus, the pressing force from the filling powder 156 weakens as F1 to F3 along the axial-line-O direction of the corners 10e. Therefore, the corners 10e having a low strength are prevented from being subjected to a high pressing force F as with the other parts (e.g., a part where the sensor element 10 abuts on the top portion 151t), whereby damage when the sensor element 10 is attached can be suppressed.

Meanwhile, in the vicinity of the top portion 151t, the distance to the sleeve 106 is short and the pressing force is high, so that the gap between the metal shell 138 and the sensor element 10 can be assuredly filled (sealed) with the filling powder 156.

Also regarding the sleeve 106, damage when the sensor element 10 is attached can be suppressed in the same manner.

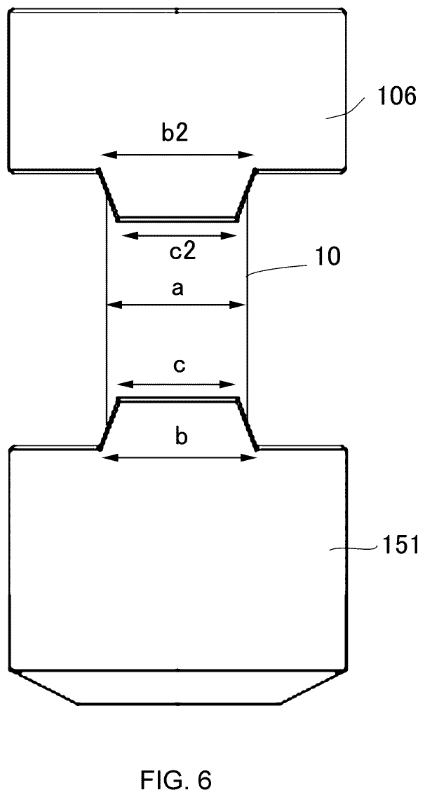

As shown in FIG. 6, in order to assuredly locate the boundary portion BR between the top portion 151t and the base portion 151b, it is preferable that a width c of the top portion 151t, a width b of the base portion 151b, and a width a of the sensor element 10 satisfy a relationship of c<a<b, as seen from the direction D.

Similarly, in order to assuredly locate the boundary portion BR between the top portion 106t and the base portion 106b, it is preferable that a width c2 of the top portion 106t, a width b2 of the base portion 106b, and the width a of the sensor element 10 satisfy a relationship of c2<a<b2, as seen from the direction D.

As shown in FIG. 3 and FIG. 4, in this example, the top portions 151t and 106t are flat (surfaces).

In this case, the filling powder 156 in a region around the sensor element 10 can be compressed at surfaces, as compared to a case where the top portions 151t and 106t have steep mountain shapes. Thus, the periphery of the sensor element 10 can be stably filled with the filling powder 156, and the gap between the metal shell 138 and the sensor element 10 can be more assuredly filled (sealed).

As shown in FIG. 4, in this example, the protrusion 106p reaches an end 106G of the flat surface 106f. Here, in the example shown in FIG. 4, the flat surface 106f extends to the outer circumferential surface of the sleeve 106 (except for a chamfer at the corner of the sleeve 106), and the outer circumferential surface corresponds to the end 106G.

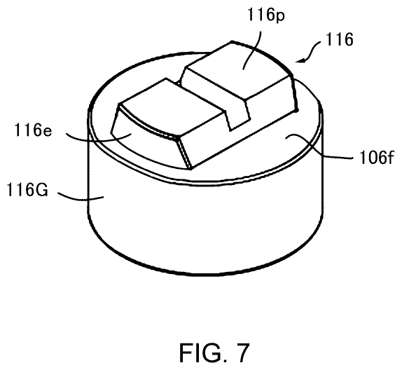

In contrast, for example, as shown in a sleeve 116 in a modification in FIG. 7, a protrusion 116p (an end surface 116e thereof) of the sleeve 116 may be located on the radially inner side relative to an end (an outer circumferential surface of the sleeve 116) 116G of a flat surface 116f.

However, when the protrusion 106p reaches the end 106G of the flat surface 106f, accordingly, the area where the filling powder 156 in a region around the sensor element 10 can be compressed with a high pressing force increases. Thus, the gap between the metal shell 138 and the sensor element 10 can be more assuredly filled (sealed).

Needless to say, the present invention is not limited to the above embodiment and includes various modifications and equivalents encompassed in the idea and the scope of the present invention.

For example, the shapes of the protrusions are not limited to those in the above embodiment.

The boundary portion may be at any position as long as the position is between the top portion and the base portion of the protrusion. The protrusion may be formed on at least one of the holder and the sleeve, but it is preferable that the protrusions are formed on both of the holder and the sleeve.

Examples of types of the gas sensor include, besides an oxygen sensor, a full-range air/fuel ratio sensor and a NOx sensor.

### DESCRIPTION OF REFERENCE NUMERALS

10 sensor element
10a detection portion
10e corner
10m1, 10m2 main surface of the sensor element
106, 116 sleeve
106h second element hole
106f, 151f flat surface
106p, 151p protrusion
106b, 151b base portion
106t, 151t top portion
106G, 116G end of the flat surface
138 metal shell
151 holder
151h first element hole
156 filling powder
200 gas sensor
O axial-line
D direction perpendicular to a main surface of the sensor element
BR boundary portion

1. A gas sensor comprising:
a plate-shaped sensor element extending in an axial-line direction and having a detection portion on a front end side thereof, said detection portion configured to detect a detection target gas;
a tubular metal shell surrounding and retaining a periphery of the sensor element in a radial direction;
a tubular holder retained in an internal hole of the metal shell and having a first element hole which has a rectangular shape and through which the sensor element is inserted;
a tubular sleeve retained in the internal hole of the metal shell on a rear end side relative to the holder, and having a second element hole, which has a rectangular shape and through which the sensor element is inserted; and
filling powder provided in the internal hole of the metal shell, between the holder and the sleeve, so as to fill a gap between the metal shell and the sensor element, wherein at least one of the holder and the sleeve has a flat surface contacting the filling powder, and a protrusion protruding from the flat surface toward the filling powder and surrounding the first element hole or the second element hole,
the protrusion is tapered toward the filling powder, as seen from a direction perpendicular to a main surface of the sensor element, and
a boundary portion, in which corners of the sensor element extending in the axial-line direction thereof abut on the protrusion, is located between a top portion of the protrusion and a base portion where the protrusion connects to the flat surface.

2. The gas sensor according to claim 1, wherein
as seen from the direction perpendicular to the main surface of the sensor element, a relationship of c<a<b is satisfied, where a is a width of the sensor element, b is a width of the base portion where the protrusion connects to the flat surface and c is a width of the top portion of the protrusion.

3. The gas sensor according to claim 1, wherein
the top portion of the protrusion is flat.

4. The gas sensor according to claim 1, wherein
the protrusion reaches an end of the flat surface.

5. The gas sensor according to claim 1, wherein
the gas sensor is an oxygen sensor or a NOx sensor.
